(19) United States
(12) Patent Application Publication
(10) Pub. No.: US 2011/0015733 A1
(43) Pub. Date: Jan. 20, 2011

(54) Folding Designs for Intraocular Lenses

(75) Inventors: Urban Schnell, Munchenbuchsee (CH); Jean-Noël Fehr, Neuchatel (CH); Alain Saurer, Neuchatel (CH); Amitava Gupta, Roanoke, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Ocular Optics, Inc., Roanoke, VA (US)

(21) Appl. No.: 12/836,154

(22) Filed: Jul. 14, 2010

Related U.S. Application Data
(60) Provisional application No. 61/225,323, filed on Jul. 14, 2009.
(60) Provisional application No. 61/250,159, filed on Oct. 9, 2009.

(51) Int. Cl. A61F 2/16

(52) U.S. Cl. 623/6.18; 623/6.43

(57) ABSTRACT

Folding patterns for intraocular lenses are provided.

### CROSS-REFERENCES TO RELATED APPLICATIONS

This application claims the benefit of U.S. Provisional Patent Applications 61/225,323 filed Jul. 14, 2009 and 61/250,159 filed Oct. 9, 2009.

### BACKGROUND OF THE INVENTION

When the natural lens of the eye becomes damaged or aged, for example, by cataract, the natural lens can be removed and replaced by an artificial intraocular lens (IOL). In many cases, the IOL is designed for monofocal distance vision, but some IOLs, such as multifocal or accommodating IOLs, may be designed to provide near vision as well.

There remains a need to provide IOLs that are surgically implantable through a small incision. There also remains a need to provide IOLs that can provide near, intermediate, and distance vision.

### BRIEF SUMMARY OF THE INVENTION

Folding designs for intraocular lenses are provided. Methods of implanting a folded intraocular lens, then unfolding the intraocular lens in vivo are also provided.

### BRIEF DESCRIPTION OF THE DRAWINGS

FIG. 1 depicts exemplary intraocular lenses, including both articulated, foldable (A, B, and C) and rollable designs (D). These designs include exemplary placement of the electronic components for the operation of the electro-active aperature.

FIG. 2 depicts an assembly view of an electronics package supporting the operation of the electro-active aperture including the batteries, the ASICs, and the antenna to support remote charging of the batteries. The electronic components can be packed onto a wafer and hermetically sealed in a thin wafer.

FIG. 3 depicts exemplary intraocular lens designs including articulated wings. A. shows hinged wings that may be used with the central full hinge design, while B. shows letterbox wings that may be used with the letterbox folding design. Both embodiments include a rigid, electro-active component. In both embodiments, the electronic components are shown at the haptic-optic junction away from the light path. In these designs, the optical sections are darkened to avoid light transmission through them while the electro-active aperture is on.

FIG. 4 depicts exemplary foldable designs for the IOL optic comprising an electro-active cell, which is mostly rigid. A. Letterbox. B. Double hinge C. Central partial hinge D. Central full hinge E. Offset single hinge. In FIG. 4, the transmissive central aperture is shown in black. The white portions (along the fold lines) are less transmissive or opaque.

FIG. 5 depicts simulated optical results for distance vision through exemplary IOL designs.

FIG. 6 depicts simulated optical results for near vision through exemplary IOL designs.

FIG. 7 depicts the Modulation Transfer Function (MTF) of the exemplary IOLs for the letter box, the central partial hinge, and the double hinge configurations. It also shows the effect on MTF at object distances of infinity (far distance) and 500 mm (intermediate distance) when the electro-active aperture is closed or open.

FIG. 8 depicts the MTF of an electro-active IOL with the electro-active aperture opened and closed as a function of object distance from infinity (90 m) to 500 mm. MTF of a retinal image with and without the aperture as a function of object distance. The plot shows that substantial improvement in MTF is seen for object distances in the range of 800 mm (0.8 M) to 5000 mm (5 M) when the aperture is ON, i.e., closed.

FIG. 9 depicts modeled folding stresses for glass at a 70° angle. A. shows a separation of 0.5 mm and a cell thickness of 100 μm resulting in 90 MPa peak stress. B. shows a separation of 0.5 mm and a cell thickness of 200 μm resulting in 27 MPa peak stress. C. shows a separation of 1 mm and a cell thickness of 100 μm resulting in 63 MPa peak stress.

### DETAILED DESCRIPTION OF THE INVENTION

The intraocular lenses (IOLs) described herein feature articulation and/or folding patterns that improve implantation and/or performance. The foldable IOLs provided herein optionally include an electro-active (EA) component, e.g., an electro-active cell, that can modify the optical power of the lens to adjust to a wide variety of visual demands including near, intermediate, and distance viewing.

In some embodiments, the electro-active component is more rigid compared to the flexible IOL body material. In one embodiment, the folding design of the IOL advantageously allows for the narrowing of the IOL profile for insertion, while minimizing or eliminating fold lines across the more rigid EA component.

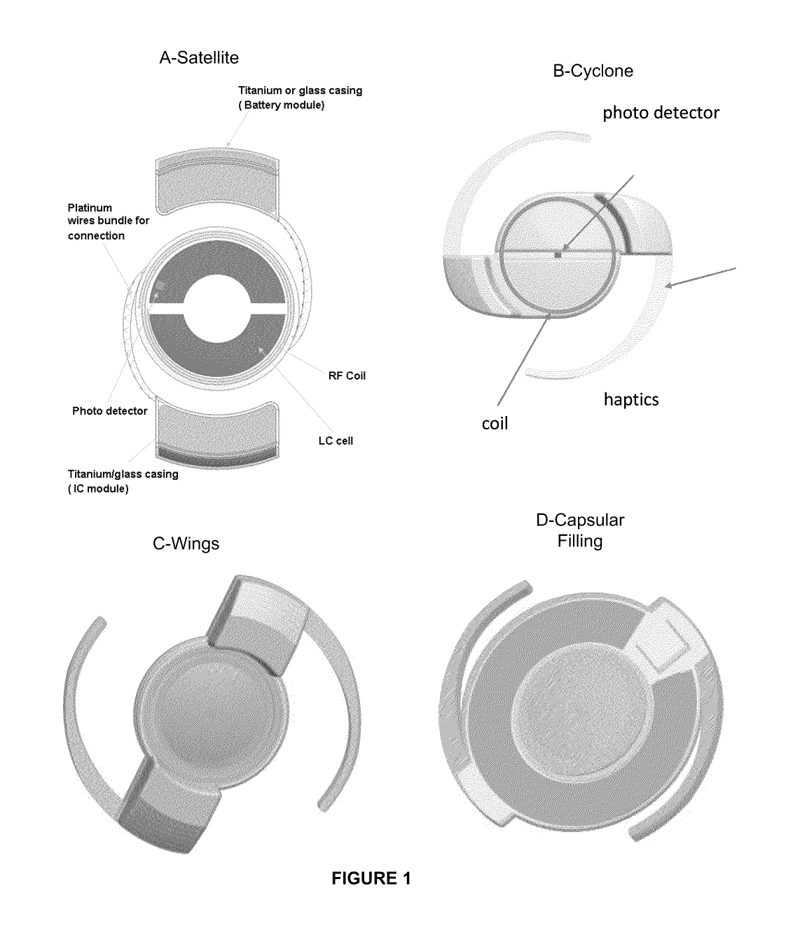

In another embodiment, the IOL may feature a flexible electronic component. The electro-active component may be fabricated out of a flexible plastic material that may be rolled in order to present a smaller profile during insertion into the eye. A flexible electro-active component may be incorporated into a rollable design, as shown in FIG. 1D. Rollable designs advantageously minimize or eliminate folding lines.

### Electronics

The IOL may include various electronic components including, but not limited to, batteries such as rechargeable batteries, a circuit such application specific integrated circuits (ASICs), antennas, and sensors. The electronic components are used to operate the electro-active component.

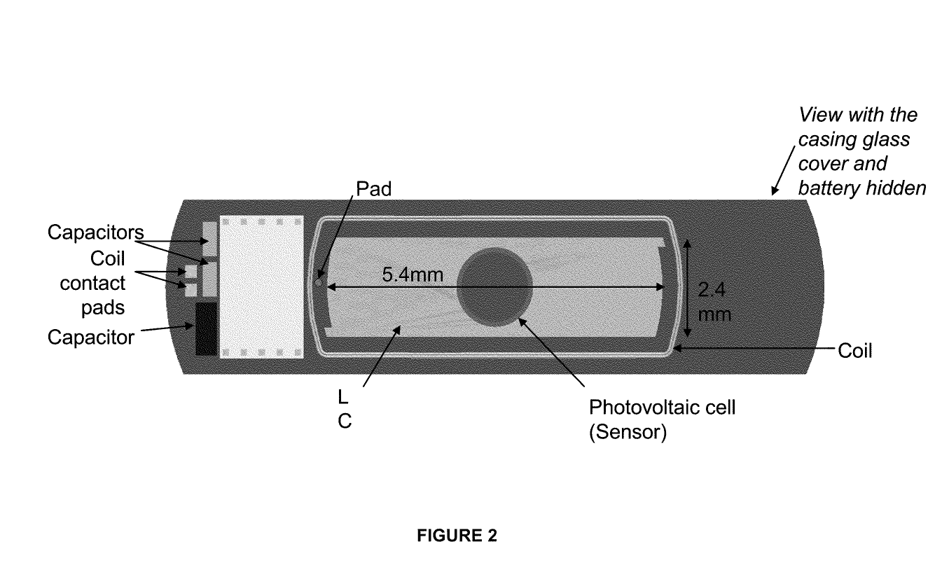

The electronic components can be grouped together or they may be spaced apart. In one embodiment, the electronic components a grouped together to form an integrated wafer. The electronics can be hermetically sealed in a thin wafer. FIG. 2 shows one embodiment of the electronic wafer that also includes the electro-active cell.

FIG. 1A shows one embodiment of a spaced apart configuration. In this embodiment, the electronic components are embedded at or near the distal edges of the haptic, while the electro-active cell remains at the center of the optic. In this configuration, an electrical connection should be provided between the electronic components and the electro-active cell.

Since the electronic components are not typically transmissive, they may be nearly anywhere on the IOL except for on the transmissive central aperture. In one embodiment, the electronic components are placed on the haptic. The electro-active aperture meanwhile may reside at the center of the optic, thus placing the electronic components away from the path of rays from objects to the retina. For example, FIG. 1A shows electronic components placed on the edges of the haptics.

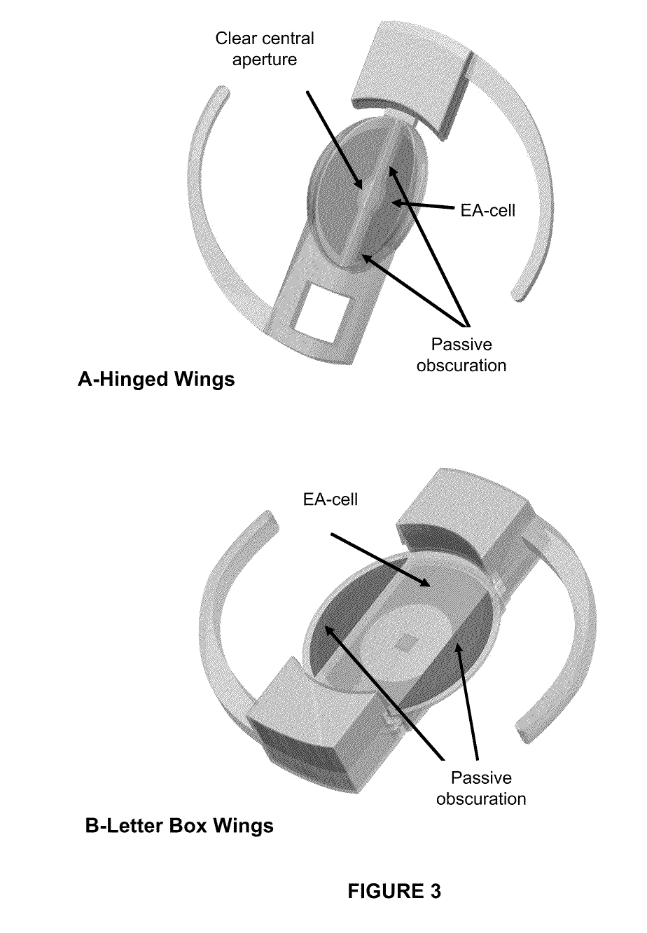

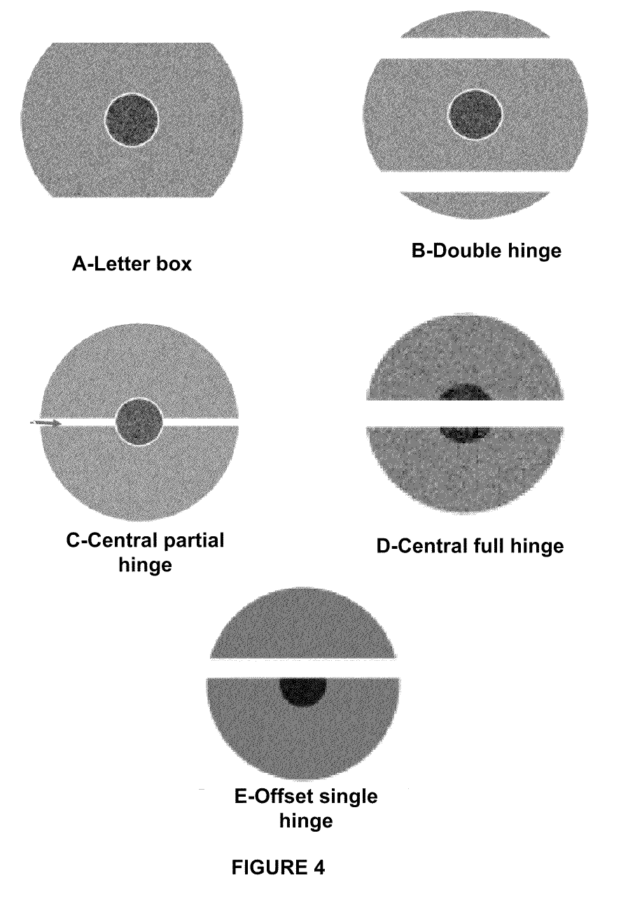

In another embodiment, the electronic components are placed at or near the haptic-optic junction. For example, they may be embedded in the hydrophobic acrylic material with at least one fold line placed such that the components that are substantially rigid do not have to be folded for the device to be implantable through a relatively smaller incision. In FIG. 1C and FIG. 3A and 3B, the electronic components are shown at the haptic-optic junction. Placement of the electronic components at the haptic-optic junction may be used with the folding designs depicted in FIG. 4.

### Folding Designs

By including strategically placed fold lines, the IOL including the EA component can be folded so that it may be inserted through a small surgical incision. Designs that incorporate such fold lines are shown in FIGS. 1, 3, and 4. FIG. 3 shows a class of designs named “wings” since it comprises a central rigid section surrounded on both sides by flexible sections that may be folded around the central rigid component. The haptics are then folded back to lie over the folded wings.

In one embodiment, an intraocular lens comprises a body comprising one or more fold lines such that the body that can assume a folded configuration and an unfolded configuration, and an electroactive component contained in or on the body, wherein at least one dimension of the folded configuration is less than about 5 mm.

The electro active component is contained on or embedded within the IOL body. In one embodiment, it is embedded within the body. The electroactive component may be constructed using materials and methods known in the art, such as in US 2006/0091528 and US 2008/0208335. The IOL may also include one or more of a battery, circuit, and sensor contained on or in the body.

The body of the IOL is constructed of a material sufficiently flexible as to allow folding to at least some degree (about 1° to about 180°, at least about 45°, or about 90° to about 180°). Exemplary materials include, but are not limited to, silicone and acrylic materials.

The IOL body may also include a transmissive central aperture. The central aperture has a transmittance of, e.g., greater than 60%, greater than 75%, greater than 90%, greater than 95%, or greater than 99%. The diameter of the central aperture is, for example, about 0.1 to about 2 mm, about 0.5 to about 1.5 mm, or about 1 mm.

The IOL described herein include one or more folding lines. The folding lines create a folding pattern, which may be symmetrical or asymmetrical across the IOL body. When the IOL is essentially planar (the folding lines are positioned at less than 10°, preferably at about 0°), the IOL is in the unfolded configuration. The unfolded configuration is also called the “in use” configuration because that is the configuration that will be assumed in vivo when in use by the wearer. When the IOL is folded along all the lines of the folding pattern, the IOL is in the folded configuration. The folded configuration is also called the “implantable” configuration because the folds reduce the dimensions of the IOL for implantation through a small surgical incision. (The IOL could be implanted in the unfolded configuration, but it would require a larger incision.) When the IOL is folded along some, but not all of the folding lines, or when the IOL is folded along one or more folding lines, but not to the degree most desirable for the implantable configuration, the IOL is said to be in a “partially folded” configuration.

The folded configuration may include folding of 180° or folding of less than 180° across one or more folding lines. Because the greater the degree of folding, the greater the internal stresses placed upon the IOL components, some embodiments are folded to less than 180°, even in the implantable configuration. In some embodiment, the IOL is folded about 1° to about 180°, about 45° to about 180°, about 70° to about 90°, about 90° to about 135°, or about 90° to about 180°. In one embodiment, the degree of folding is any degree that results in a peak stress of less than about 70 MPa, less than about 65 MPa, less than about 60 MPa, less than about 50 MPa, less than about 40 MPa, less than about 30 MPa, or less than about 25 MPa. These peak stress levels can be assessed at the surface of the IOL, within the IOL body, and/or between cells.

In one embodiment, the fold line can have a width (hinge size) of about 0.1 mm to about 1 mm, about 0.25 to about 0.75 mm, about 0.3 mm to about 0.8 mm, about 0.5 mm to about 0.6, or about 0.5 mm. This measurement assesses the portion of the IOL that is under fold stress as opposed to the remainder of the IOL that remains substantially planar even in the folded configuration.

In one embodiment, the thickness of the IOL body is about 0.1 to about 2 mm, about 0.5 to about 1.5 mm, or about 1 mm.

In another embodiment, the thickness of the electroactive component is about 50 μm to about 500 μm, about 100 μm to about 300 μm, about 150 μm to about 250 μm, or about 200 μm or less.

The fold lines can transmit or absorb light. For example, the fold line can have a transmittance of greater than 99%, greater than 95%, greater than 90%, about 70% to about 90%, about 50% to about 75%, about 30% to about 50%, less than about 20%, less than about 10%, or less than about 5%. When the fold lines are designed to transmit light, they are designed to minimize distortion of light rays transmitted by them when the IOL is in position inside the capsular sac. Thus, in one embodiment, a fold line has a transmittance of at least 90%. When distortion of light rays transmitted through the fold lines cannot be avoided, the fold lines are rendered less transmissive or opaque to avoid introducing distorted rays on the retina. Thus, in another embodiment, a fold line has a transmittance of less than 20%.

In one embodiment, the folding pattern include two parallel fold lines. In one embodiment, the distance between each folding line to the closest outer edge of the IOL body is the same, such that the fold lines divide the generally circular IOL into two equal segments and a center portion. Exemplary folding patterns of this type include the letterbox pattern shown in FIG. 4A and the double hinge pattern shown in FIG. 4B. In another embodiment, the distance between each folding line to the closest outer edge of the IOL body is also the same as the distance between the folding lines, such that the segments and the center portion all have the same width.

In a preferred embodiment, the IOL includes a letterbox folding pattern, where the IOL is folded along two parallel folding lines to greater than 90°, greater than 135°, or about 180°. In one embodiment the IOL is folded along the folding lines to about 180°, such that the IOL is folded like a tri-fold letter for insertion into an envelope. The letterbox design allows the placement of all electronic components required to drive the electro-active aperture at the haptic-optic junction out of the path of light rays being focused by the IOL. It also allows the substantially rigid electronics package including the electro-active aperture to remain unfolded while folding the IOL to a size that is capable of being implanted through an incision smaller than 5 mm.

In another embodiment, the IOL includes a double hinge folding pattern, where the IOL is folded along two parallel folding lines to about 30° to about 90°, about 45° to about 90°, or about 90° or less. In one embodiment, the IOL is folded along the folding lines to about 90°.

In one embodiment, at least one fold line that traverses the central aperture. Exemplary folding patterns of this type include the central full hinge shown in FIG. 4D and the offset single hinge shown in FIG. 4E. In one embodiment, at least one folding line bisects the IOL body, i.e., the folding line traverses the center point of the IOL. Exemplary folding patterns of this type include the central partial hinge shown in FIG. 4C and the central full hinge shown in FIG. 4D. Folding lines that traverse the central aperture may or may not require the folding of the central aperture. In some embodiments, the folding line extends fully across the IOL body through the central aperture. In other embodiments, the folding line may be discontinuous as in the central partial hinge pattern of FIG. 4C.

In one embodiment, the central aperture remain substantially planar in both the folded and unfolded IOL configuration. This can be accomplished by, e.g., 1) a folding pattern in which the folding line(s) do not traverse the central aperture, or 2) a folding pattern including a discontinuous folding line that traverses the central aperture.

In general, the folding patterns described herein permit the IOL to be implanted through a surgical incision that is less than about 5 mm. Because the IOL body is generally about 6 mm in diameter (or about 12 mm including haptics), the folding permits a smaller incision that would be required to insert the IOL in the “in use” configuration. Accordingly, in one embodiment, the folded configuration includes a dimension that is less than about 5 mm, less than about 4 mm, less than about 3.5 mm, less than about 3 mm, less than about 2.5 mm, or less than about 2 mm. In one embodiment, the folded configuration includes a dimension that is 3.5 mm or less. In another embodiment, the folded configuration includes a dimension that is about 3.2 to about 3.5 mm. These size parameters for the folded IOL directly relate to the surgical incision size for the methods of implanting an IOL, discussed in further detail below.

The IOL may also include haptics to secure the IOL in place in vivo. Arrangement and design of haptics is well known in the art. In some embodiments herein, the IOL includes articulated haptics. The haptics (typically two) may extend concentrically to the circumference of the generally circular IOL body. In one embodiment, the IOL further includes wings that space the haptics away from the outer edge of the IOL body. The wings may be flexibly connected to the IOL body such that they may hinge and/or pivot relative to the IOL body. See FIG. 3.

### Methods of Implanting Foldable IOLs

In another embodiment, a method of implanting an intraocular lens includes the steps of: providing a foldable intraocular lens as described herein above; providing the intraocular lens in a folded configuration; inserting the folded intraocular lens into the eye; and unfolding the intraocular lens into its unfolded configuration.

In one embodiment, inserting the folded IOL into the eye includes inserting the folded IOL through a surgical incision that is less than about 5 mm, less than about 4 mm, less than about 3.5 mm, less than about 3 mm, less than about 2.5 mm, or less than about 2 mm. In one embodiment, the surgical incision is 3.5 mm or less. In another embodiment, the surgical incision is about 3.2 to about 3.5 mm.

Unfolding the IOL can include actively unfolding the IOL or passively permitting the IOL to assume its unfolded state (depending on the resiliency of the IOL material).

### Analysis of Image Quality

The foldable IOLs provided herein can provide exceptional vision performance despite the folding disruption. The following measures of vision performance are achieved after folding and unfolding of the IOL.

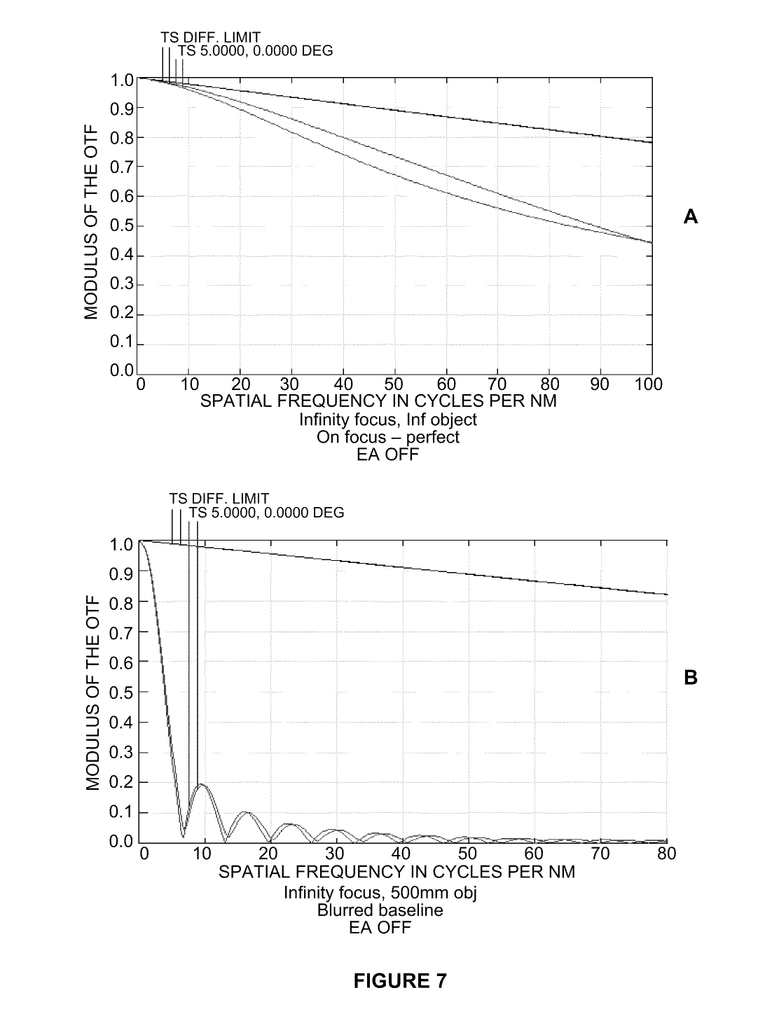

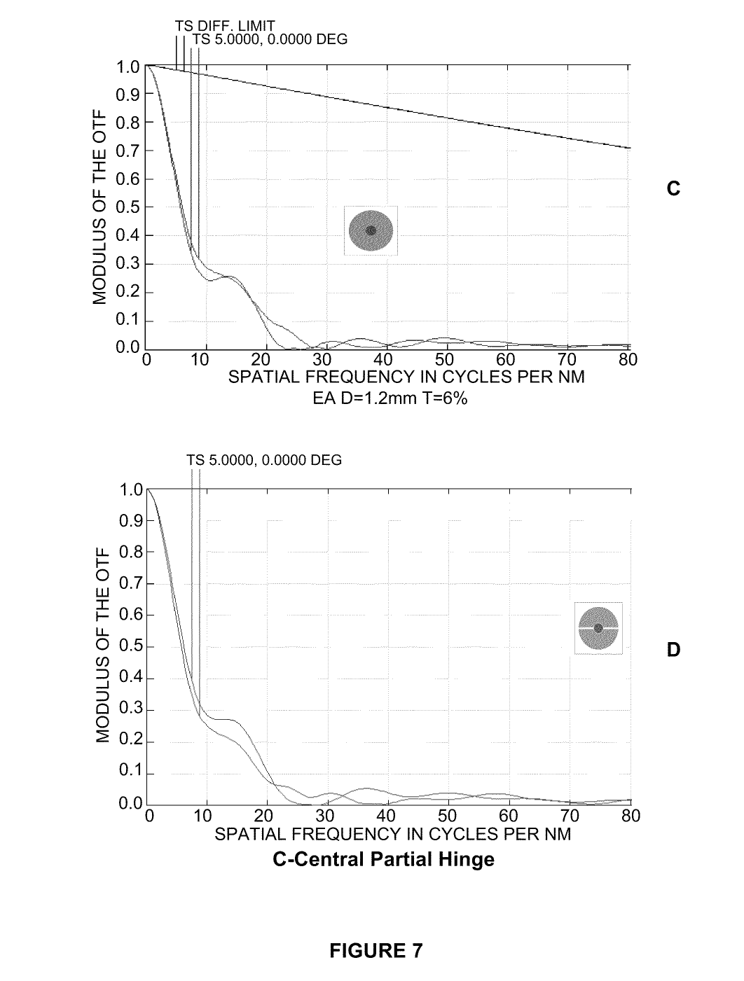

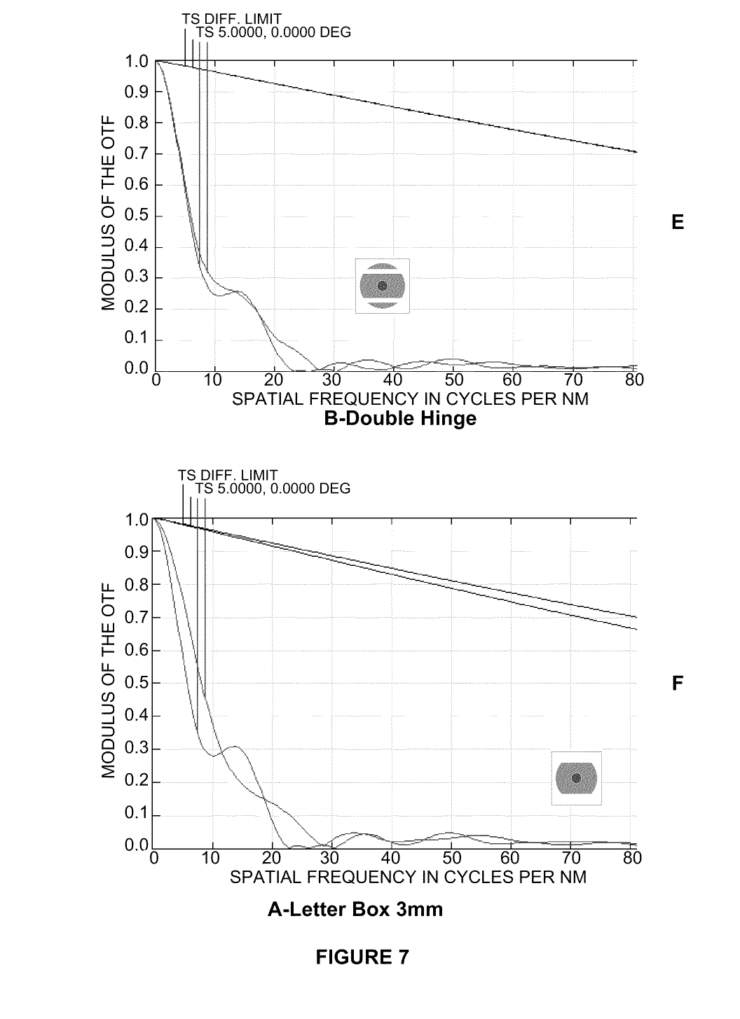

In one embodiment, the IOL achieves a modulation transfer function (MTF) of at least about 5%, at least about 10%, at least about 15%, at least about 20%, or at least about 25%. See FIG. 7. In one embodiment, the IOL achieves this MTF for distance, intermediate, and/or near vision focal tasks. In one embodiment, the IOL achieves this MTF for near vision. In another embodiment, the IOL achieves this MTF for intermediate vision. In another embodiment, the IOL achieves this MTF for distance vision. In yet another embodiment, the IOL achieves this MTF for all of near, intermediate, and distance vision.

### EXAMPLES

### Example 1

### Optical Modeling

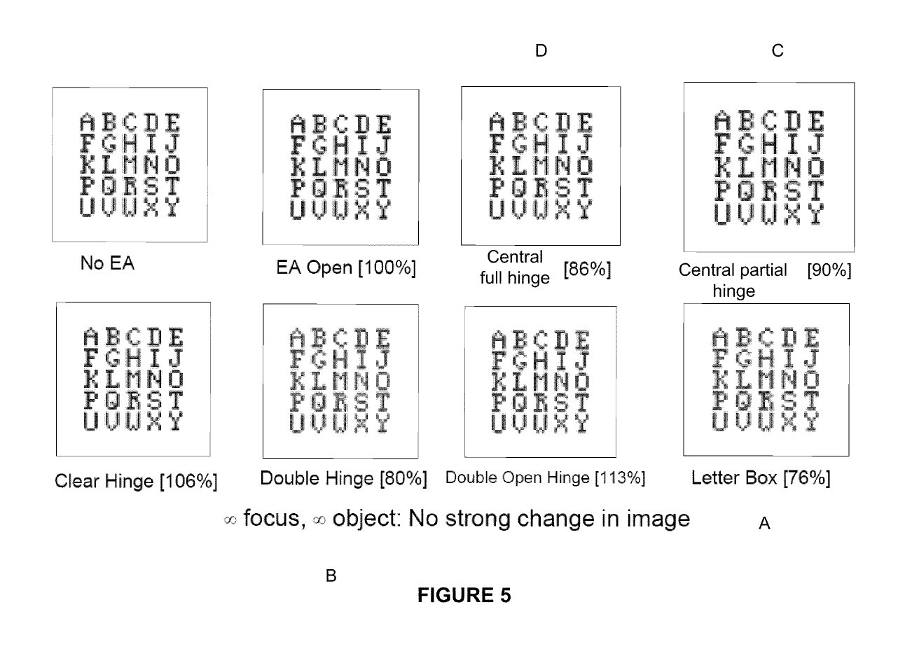

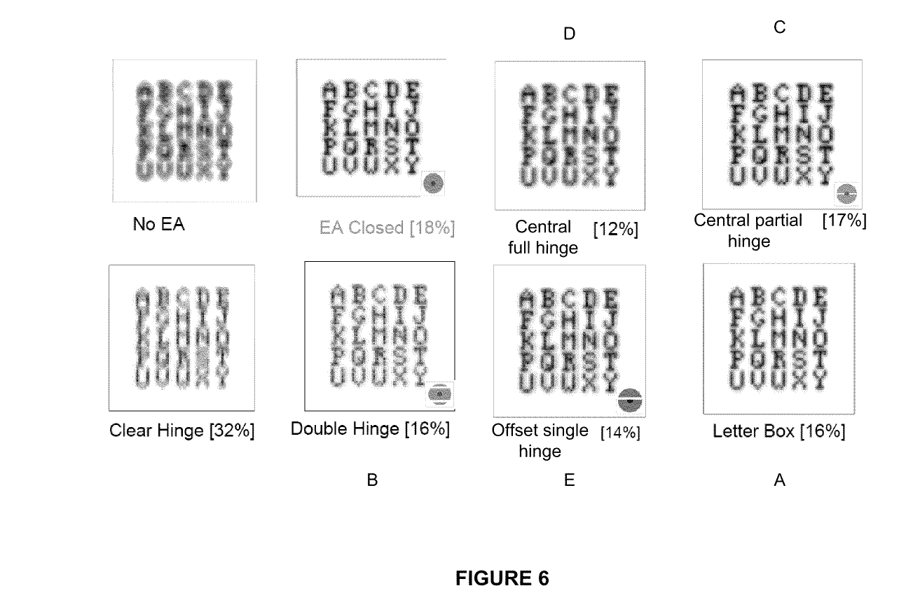

Optical performance was assessed by analyzing image quality of exemplary folded IOL designs modeled using Liou Brennan eye model in ZEMAX® software. The results are shown in FIGS. 5-6.

As shown in FIG. 5, distance vision was substantially maintained with the exemplary folded IOLs. Near vision is improved by adding the EA cell, when the cell is turned ON. The extent of improvement depends on the mechanical design of the IOL, and is found to be the best for the letterbox design.

### Example 2

### Modulation Transfer Function

The Modulation Transfer Function (MTF) of several exemplary IOLs (central partial hinge, double hinge, and letterbox) were simulated and compared to a control system with no fold lines. The MTF was simulated for an object at 500 mm while the electroactive component was set to focus at infinity.

As shown in FIG. 7, the exemplary IOLs demonstrate a significant improvement in near vision.

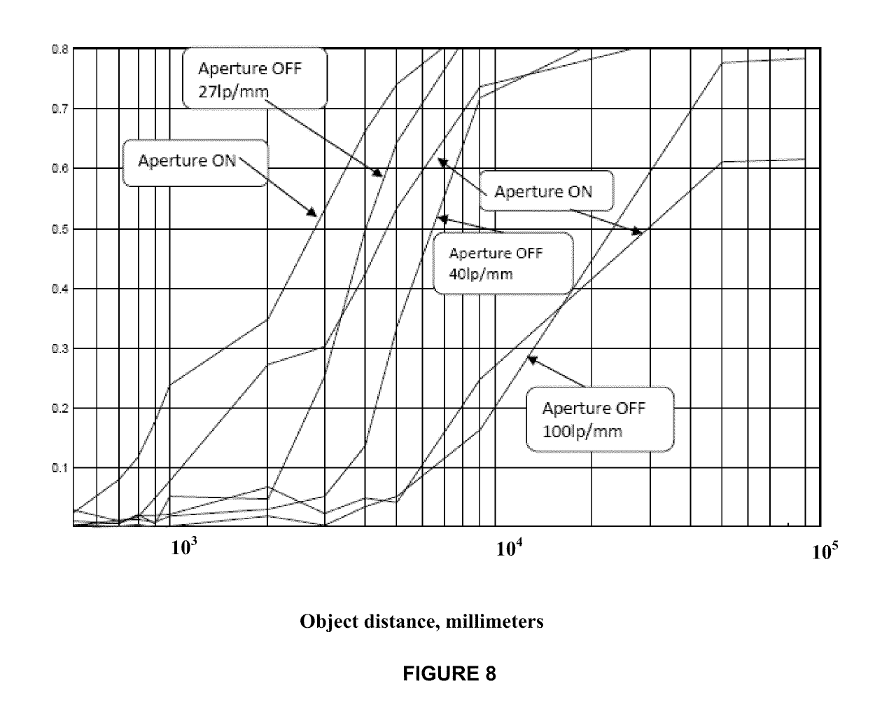

Next, the MTF was simulated while varying the object distance from infinity (90 m) to 500 mm to assess vision at intermediate distances. The MTF for 100 line pairs/mm (as used for ISO 11979-2), 40 Ip/mm, and 27.5 Ip/mm can be seen to improve as the liquid crystal transmission is varied from 60% (clear) to 6% (opaque). See FIG. 8.

### Example 3

### Stress Tests

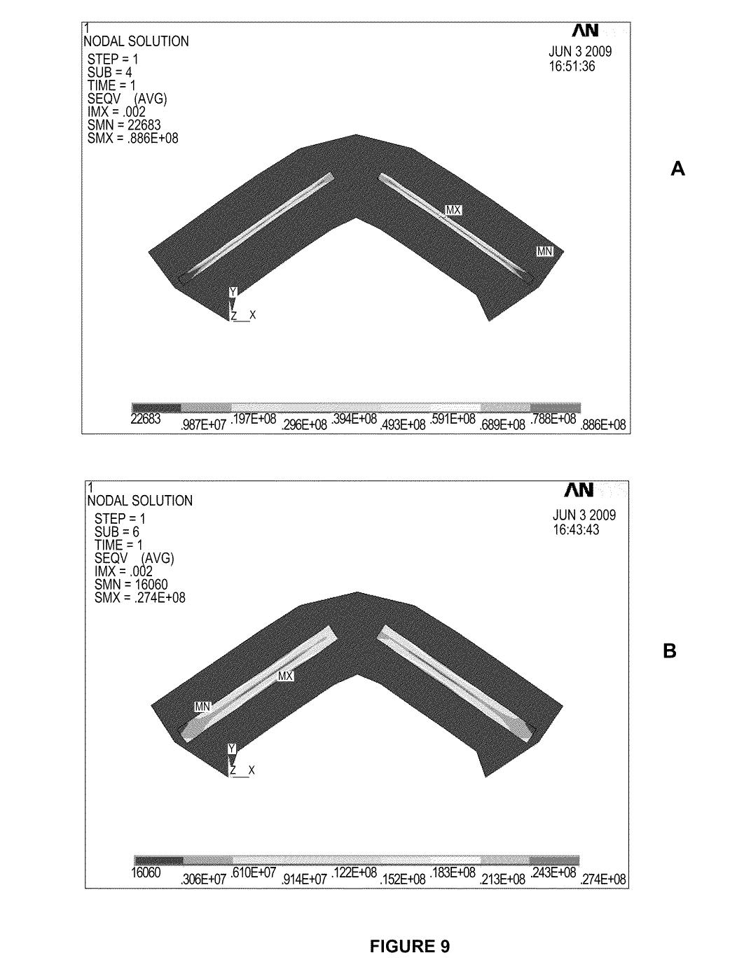

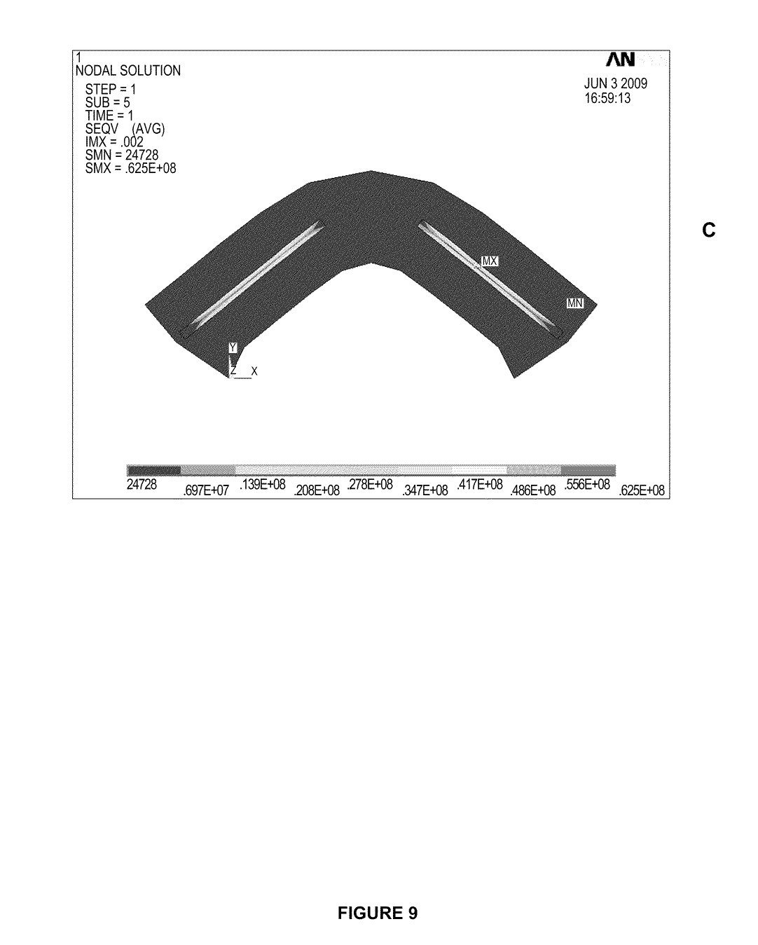

To create foldable IOLs, the glass components must be able to withstand a certain amount of folding stress. Glass stress tests were modeled. The variable parameters and the resulting peak stresses are shown in FIG. 9 and provided below:

| | Separation | Cell thickness | Peak Stress |
|---|---|---|---|
| Model | (mm) | (μm) | (MPa) |
| A | 0.5 | 100 | 90 |
| B | 0.5 | 200 | 27 |
| C | 1 | 100 | 63 |

For a 1 mm thick lens, the glass preferably exhibits a peak stress of less than 70 MPa. As demonstrated by these modeled stress tests, glass stress may be reduced by increasing the separation (compare models A and C) and/or by increasing cell thickness (compare models A and B).

What is claimed is:

1. An intraocular lens comprising:
a body comprising one or more fold lines such that the body that can assume a folded configuration and an unfolded configuration, and
an electroactive component contained in or on the body,
wherein at least one dimension of the folded configuration is less than about 5 mm.

2. The intraocular lens of claim 1, comprising two parallel fold lines.

3. The intraocular lens of claim 1, comprising a fold line that traverses the central aperture.

4. The intraocular lens of claim 1, comprising a fold line that bisects the body.

5. The intraocular lens of claim 1, wherein the central aperture remains substantially planar in both the folded and unfolded configurations of the intraocular lens body.

6. The intraocular lens of claim 1, wherein at least one dimension of the folded configuration is less than about 4 mm.

7. The intraocular lens of claim 6, wherein at least one dimension of the folded configuration is less than about 3.5 mm.

8. The intraocular lens of claim 7, wherein at least one dimension of the folded configuration is less than about 3 mm.

9. The intraocular lens of claim 1, wherein the intraocular lens, having been folded and unfolded, achieves a modulation transfer function of at least about 5%.

10. The intraocular lens of claim 1, wherein the intraocular lens further comprises articulated haptics.

11. The intraocular lens of claim 10, further comprising wings flexibly connected to the body, wherein the haptics extend concentrically around the perimeter of the body from the wings.

12. The intraocular lens of claim 11, wherein the wings can hinge and/or pivot relative to the body.

13. The intraocular lens of claim 1, wherein the intraocular lens further includes one or more electronic components selected from the group consisting of a battery, a circuit, an antenna, and a sensor.

14. The intraocular lens of claim 13, wherein one or more electronic components is positioned on a haptic.

15. The intraocular lens of claim 13, wherein one or more electronic components is positioned on a haptic-optic junction.

16. A method of implanting an intraocular lens comprising:
providing an intraocular lens as in claim 1;
folding the intraocular lens into the folded configuration;
inserting the folded intraocular lens into the eye;
unfolding the intraocular lens into its unfolded configuration.
